(19) United States
(12) Patent Application Publication
(10) Pub. No.: US 2016/0113978 A1
(43) Pub. Date: Apr. 28, 2016

(54) A PHARMACEUTICAL COMPOSITION FOR THE PROPHYLAXIS AND THE TREATMENT OF DISEASES OF INFECTIOUS AETIOLOGY

(71) Applicant: Giorgio SCAPECCHI, Lugano (CH)

(72) Inventors: Bernard Bizzini, Carcassonne (FR); Laura Ferrari, Marina di Carrara (IT)

(21) Appl. No.: 14/894,960

(22) PCT Filed: May 30, 2014
(86) PCT No.: PCT/IB2014/061846

§ 371 Date: Nov. 30, 2015

(30) Foreign Application Priority Data
May 31, 2013 (IT) FI2013A000131

(51) Int. Cl. A61K 36/064; A61K 35/747

(52) U.S. Cl. CPC A61K 36/064; A61K 35/747

(57) ABSTRACT

A pharmaceutical composition for the topical or parenteral administration useful for the prophylaxis and treatment of diseases with infectious aetiology, both viral and bacterial diseases, in particular diseases affecting the oral cavity, such as gingivitis, periodontitis and perimplantitis.

### FIELD OF THE INVENTION

The present invention relates to the field of pharmaceutical products, and more specifically it provides a pharmaceutical composition useful in the prophylaxis and treatment of infections, in particular of diseases having infectious aetiology involving the oral cavity, such as periodontitis, gingivitis and peri-implantitis.

### STATE OF THE ART

The diseases of the oral cavity are widespread all over the world and do represent a general problem for the health of the entire body. In many chronic diseases, in fact, like diabetes, cardio-vascular diseases, cancer, just to cite a few, an influence on these diseases has been found that is exerted by infections of the oral cavity. This is explained, at least partially, by the fact that at the level of the oral cavity, as well as at the level of the intestine, a microbial flora prevails whose imbalance is expressed through dysfunctions and diseases, because this microbial flora plays an important role in the protection from the installation of pathogenic microbes.

In particular, among these diseases, the periodontal disease is an important one: by this term are commonly indicated all diseases that are characterized by an inflammation of the tissues around the teeth and by their degeneration, that may have only a superficial development, giving rise to or gingivitis, or they may progress to periodontitis or involve instead only the deep periodontium, appearing in an acute or chronic or recurrent form. These diseases have a multifactorial aetiology, polymicrobial, where the lack of hygiene leads to an infection by pathogenic microbes, probably explainable with a weakening of the body's natural defences, or with an increase in the load of infection or of the free radicals at this level.

Saccharomyces cerevisiae is a fungus whose fermentation produces alcohol; it has been used since ancient times in the production of wine and beer and in bread-making, and it is also one of the most studied microorganisms for medical applications. Up from 800, by noting that the employees of breweries were never suffering from furunculosis, it was hypothesized that the brewer's yeast should exert an important therapeutic activity and, starting from 1852 the brewer's yeast, obtained from cultures of selected strains of Saccharomyces cerevisiae, was used for the treatment of infectious diseases in Western countries and, shortly after, the use thereof for the treatment of bacterial infections was extended to the whole world.

Many authors reported of experimental data showing the ability of the cells of Saccharomyces cerevisiae to enhance the non-specific mechanisms of natural immune defence, for instance Bizzini B. et al. FEMS Microbiol. Immunol., 1990, 64, 155-168, that showed how the cells of Saccharomyces cerevisiae increase resistance in mice to infections caused by Klebsiella pneumoniae, Streptococcus pneumoniae and Streptococcus pyogenes induced by intranasal route, to infections by Lysteria monocytogenes and Salmonella typhimurium caused by intravenous route, and to the viral infections caused by HSV-1 (Herpes simplex virus-1) and by the influenza virus.

Lactobacilli are a genus of Gram-positive bacteria, also present in the human body, and in general they are non-pathogenic. They produce in particular lactic acid and other acidic species by fermentation of sugars, thus reducing the pH of the environment in which they grow and inhibiting, thanks to the acidification of the environment, the growth of certain pathogenic microorganisms. Experimentally, it has been highlighted the ability of lactobacilli to exert an adjuvant effect (Blocksma K. et al., Exp. Immunol., 1979, 37, 367-375) and to have antitumor activity (Battazzi V. et al., II latte, 1985, 10, 878-879), to increase resistance against infections by L. monocytogenes (Katsumasa et al., Infect. Immun., 1984, 44, 1445-1451) and by Herpes Simplex virus (T. Watanabe et al., Microbiol. Immunol., 1986, 30, 111-112), as well as their ability to activate macrophages (see for instance Kato I. et al., Microbiol. Immunol., 1983, 27, 611-618).

Macrophages are tissue cells belonging to the phagocyte system, and play a very important role in innate and specific immune responses: their primary function is phagocytosis, which is the ability to embed in the cytoplasm foreign particles, such as micro-organisms, and to destroy them. In addition to this action of defence against microbes, macrophages also play an antiviral action, producing cytokines able to inhibit the replication of viruses, and therefore the spread of the virus to the healthy cells.

To restore the macrophage in its functions when it is debilitated, or to activate it when it has to deal with a particularly virulent microbe, chemical substances may be administered, that are obtained by purification from bacteria or by chemical synthesis, and are generically referred to as “immunomodulators”. The immunomodulatory effect is also due to the ability to induce the formation of interferon (IFN); in particular, it was verified that the immunomodulatory agent induces both the formation of IFN-alpha and IFN-gamma in whole blood and in cultures of peripheral blood lymphocytes (PBL). The formation of cytokines, along with the activation of complement, also explains, at least in part, the non-specific effects of immunostimulation.

As far as the Applicant is aware of, are not known to date pharmaceutical compositions of Saccharomyces cerevisiae and Lactobacilli useful in the treatment and prevention of infections, in particular of the oral cavity.

### SUMMARY OF THE INVENTION

The Applicant has now surprisingly found that the cellular extract of Saccharomyces cerevisiae and of Lactobacillus acidophilus, if associated in a single composition, are able to synergistically act against infections, both bacterial and viral infections, by activating and strengthen the functions of macrophages; they have also proved capable of achieving a protection from infections in immunosuppressed conditions, of inducing a hypersensitivity towards various antigens, and of achieving an effective immunomodulation.

In particular, the association of the above said cellular extracts, appropriately formulated for the topical administration on the mucosa of the oral cavity, has proved capable of treating diseases having infectious aetiology, such as gingivitis, pyorrhoea, periodontitis and perimplantitis, and to prevent their onset.

Thanks to these features, the mixture of cellular extracts of Saccharomyces cerevisiae and of Lactobacillus acidophilus according to the invention may be used in pharmaceutical compositions useful for the treatment and prevention of diseases of infectious aetiology, in particular of the above said diseases that affect the oral cavity.

Subject of the present invention is therefore a pharmaceutical composition for the treatment and prophylaxis of diseases having infectious aetiology, in particular of those diseases that affect the oral cavity, comprising as active principle a mixture of cellular extracts of Saccharomyces cerevisiae and of Lactobacillus acidophilus.

A further subject of the invention is the use of mixtures of cellular extracts of Saccharomyces cerevisiae and of Lactobacillus acidophilus, for the treatment of diseases of infectious aetiology, in particular of infections of the oral cavity.

Still a further subject of the invention is a process for the preparation of the above said pharmaceutical composition.

Further features and advantages of the invention will appear more clearly from the following detailed description.

### DETAILED DESCRIPTION OF THE INVENTION

The compositions of the present invention have proven capable of activating and stimulating the macrophage function in the immune response and of increasing the resistance to infections, both bacterial and viral infections. Furthermore, the present compositions are able to achieve a protection from infections in immunosuppressed conditions, to induce a hypersensitivity to various antigens, and to achieve an effective immunomodulation.

The compositions of the invention comprise as active principle a mixture of cellular extracts of Saccharomyces cerevisiae and Lactobacillus acidophilus, formulated with pharmaceutically acceptable excipients, diluents and/or stabilizing agents, selected from those that are commonly used in the field depending on the type of formulation, which is desired for a certain type of administration.

The preferred administration forms of the present compositions are all the forms suitable for topical administration, typically creams, ointments, pomades, gels, solutions, such as for mouthwash, and similar, and for parenteral administration, typically solutions, suspensions, granules and powders. The preferred administration way of the present compositions for the treatment of infections of the oral cavity is the topical way, in particular in the form of a gel or of a mouthwash.

The content of the cellular extracts of Saccharomyces cerevisiae and of Lactobacillus acidophilus in the present compositions is defined so as to allow for the best efficacy, for instance the amount of the two extracts in the mixture is the same or approximately the same, whereas the content of the mixture in the final composition for instance may be comprised between 10 and 200 μg per gram of excipients, diluents and stabilizing agents, or anyway per gram of the remaining components in the composition. The preferred amounts of the two extracts vary depending on the formulation of the composition itself, which on its turn varies depending on the particular application to which it is intended; for instance, for compositions intended for the use in the treatment and prevention of diseases of the oral cavity, that are preferably formulated in the form of a toothpaste, of a mouthwash or of a gel, preferred amounts of the mixture of two extracts are 25 μg for the mouthwash, 10 μg for the toothpaste, and 25, 50, 80 or 100 μg for the gel composition, all always per gram of the remaining components in the composition. These latter amounts are furthermore preferred amounts also in the compositions of the invention formulated as pomade or nasal spray, for the topical administration in other types of diseases having infectious aetiology hereinafter specified.

The present compositions may further comprise other active principles, having for instance stimulating activity of the immune system and/or treatment of infections.

The compositions of the invention are particularly indicated for the prophylaxis and treatment of infectious diseases that affect the oral cavity, such as gingivitis, pyorrhoea, periodontitis, perimplantitis, and periapical lesions, for this indication particularly preferred are the compositions for the topical administration, for instance in the form of mouthwash, of toothpaste or of gel for the direct contact of the active principle with the mucosa affected by the disease. Always in relation to the diseases of the oral cavity, the present compositions are useful for the prevention of caries, for the treatment of halitosis and wounds of the skin and of the mucous membranes and as coadjuvants for use in therapies muco-gingival.

Further applications of the present compositions are in the treatment of infections from Candida albicans, of vulvo-vaginitis, of onychomycosis, of cutaneous mucosal lesions due to Herpes Virus Simplex 1 and 2, of pressure ulcers and diabetic ulcers, of tonsillitis and pharyngitis, of recurrent infections of the urethra, of the lower urinary tract or of the respiratory system, of infective forms in immunocompromised patients or in hospitalized elderly patients, of microtrauma to the cornea in subjects with contact lenses, of allergic rhinitis, of folliculitis, lesions of acne, chapped and excoriated skin, of wrinkles and stretch marks, as well as in the treatment of inflammatory microenvironment.

The subjects that may be treated with the pharmaceutical compositions of the invention can be humans and animals, such as dogs, cats or horses.

The mixture of cell extracts according to the invention may be prepared by wet mixture of the two cell extracts separately obtained by homogenization of aqueous suspensions of Lactobacillus acidophilus cells and by homogenization of aqueous suspension of Saccharomyces cerevisiae cells followed by extraction with sodium hydroxide.

The following examples are provided to illustrate the present invention without limiting it.

Chemical Experimental Part

### Example 1

### Preparation of the Extract of Cell Walls of Saccharomyces Cerevisiae

An extract of cell walls of Saccharomyces cerevisiae has been obtained as follows. An aqueous suspension has been prepared with 10 g of cells in 100 ml of water; the suspension has been then subjected to crushing with a homogenizer Ultraturrax® at the maximum speed for 5 min, then cooled with ice, in order to isolate the cell walls. The cell walls have been recovered by centrifugation, washed with water twice, every time recovering the material by centrifugation, then re-suspending it in water.

The washed cell walls have been treated with a solution 1 N of NaOH, at 50° C. and after cooling the insoluble fraction was sedimented by centrifugation, and the supernatant was collected and neutralized by addition of HCl 5 N (first extract). The sediment was washed several times with water before treatment with boiling HCl 6 N. The insoluble fraction has been sedimented by centrifugation and the supernatant was collected and neutralized by addition of NaOH 10 N (second extract). The two extracts have been mixed and dialysed against water in a tube of size 1 KD.

### Example 2

### Preparation of the Extract of Lactobacillus Acidophilus

The extract of cell walls of Lactobacillus acidophilus was prepared by application of the following procedure.

Lactobacillus acidophilus was cultured in a nutrient medium enriched in glucose, at 37° C., for 36-48 hours. After growth, the bacteria were collected by centrifugation and washed 2 times by centrifugation after re-suspension in water. A suspension of bacteria in water (10 g of wet weight per 100 ml) was subjected to a rupture in a homogenizer Waring Blender for 3 cycles of one minute each, with ice-cooling. After crushing, the unbroken bacteria were eliminated by centrifugation at 1000 xg for 5 min. From the supernatant the walls were sedimented at 10,000 xg for 15 min. After re-suspension in water, the cell walls were washed two times by centrifugation.

### Example 3

### Preparation of the Composition of the Invention

The composition of the invention was prepared by mixing the extract from the cell walls of Saccharomyces cerevisiae obtained as described above in Example 1 with the extract of the cell walls of Lactobacillus acidophilus obtained as described above in Example 2, in the ratio of 1 mg of dry weight of each of these extracts. The mixture obtained was concentrated to dryness in a rotary evaporator under vacuum, and the dry residue was then collected and ground in a mortar.

Analytical Experimental Part

In the pharmaceutical composition of the invention the extract of the walls of yeast contributes with mannan and glucan. Thus, the hydrolysis of the composition of the invention with 4 N HCl for 4 hours at 100° C. results in the release of glucose and mannose, which can be titrated by the colorimetric method with the anthrone reagent. On the other hand, the separation of glucose from mannose can be carried out by chromatography on thin layer of silica gel (Hansen S. A. J. Chromatog., 1975, 107, 224-226). In the present composition the presence of the walls from Lactobacillus acidophilus was instead confirmed by hydrolysis with HCl 6N, at 105° C., for 15 hours that highlights the presence of diaminopimelic acid by cellulose thin layer chromatography (Primosigh J. et al. Biochim. Biophys. Acta, 1961, 45, 65-80).

Determination of the Ability of the Present Composition to Activate Macrophages

For this determination lots of 6 Swiss mice were used, 18-20 g of body weight. Four of these mice received intravenously 400 μg of the composition of the invention in 100 μl of saline solution. For the remaining two mice, that were the controls, 100 μl of saline solution were administered intravenously. After 5 days, from all mice blood was collected by intra-cardiac puncture.

From the blood the polymorphonuclear neutrophils (PMNs) were isolated, and their ability to ingest cells of Salmonella typhimurium was evaluated. The results showed that the PMNs isolated from mice treated with the present composition had ingested a quantity of bacteria approximately 4 times larger compared to the PMNs isolated from the controls. The treatment with the present composition has therefore given rise to a strong activation of macrophages.

Determination of the Ability of the Present Composition to Increase Resistance to Bacterial Infections in Mice

Lots of 8 Swiss mice were used, 4 to be treated and 4 as controls. To the treated mice a gel containing 50 μg of the present composition was smeared on the nasal mucosa, for 3 times with a day of interval between a treatment and the next one; controls were instead smeared with the same gel but without the present composition. All mice were then infected by intranasal instillation of 10 μl of a suspension of Klebsiella pneumoniae with a concentration of 3×10⁶ CFU/ml. The protective effect of the present composition is evident from the survival data observed, with 3 out of 4 treated mice and only 1 control mice out of 4, survived to bacterial infection.

Clinical Experimental Part

1) Topical treatment of 10 volunteer subjects with gingivitis and/or acute periodontitis:

10 volunteer subjects have smeared their gums, for 5 days, twice a day, with a dose of gel containing 25 μg of the present composition. The progress of the disease was observed daily. In subjects with gingivitis a significant improvement of the inflammatory process was already noted after 24 hours from the start of treatment, and the absence of any inflammatory phenomenon starting from the second day. In the case of subjects who were initially affected by periodontitis, there was already an improvement in their condition starting from the second day, with the disappearance of all clinical signs on the fifth day after the start of treatment.

2) Prevention of recurrence of periodontal disease by use of a preparation containing the composition of the invention

In 15 volunteers with recurrent periodontal disease, the daily use of a preparation containing the composition of the invention (50 μg of composition per gram of preparation) led to the absence of any recurrence during the observation period of 3 months.

Moreover, the treatments carried out with the present composition have not given rise to side effects, resulting perfectly tolerated.

The present invention has been described with reference to a preferred embodiment thereof. It should be understood however that other embodiments may exist belonging to the same inventive core, as defined by the scope of protection of the following claims.

1. A pharmaceutical composition for the topical or parenteral administration in the treatment of patients suffering of diseases of infectious aetiology or in the prophylaxis of said diseases, comprising as active principle a mixture of cellular extracts of Saccharomyces cerevisiae and of Lactobacillus acidophilus, alone or in combination with other active principles.

2. The composition according to claim 1, further comprising one or more pharmaceutically acceptable excipients, diluents and/or stabilizing agents suitable for the topical or parenteral administration of the composition, and comprising said mixture in amount ranging between 10 and 200 μg per gram of excipients, diluents and/or stabilizing agents in the composition.

3. The composition according to claim 1, for use in the treatment and prophylaxis of diseases of the oral cavity such as: gingivitis, pyorrhoea, periodontitis, peri-implantitis, periapical lesions, dental caries, halitosis, and cutaneous-mucosal wounds.

4. The composition according to claim 3, in the form of a preparation, a toothpaste, or a gel for the use in the prophylaxis and treatment of gingivitis, pyorrhoea, and periodontitis, in humans and animals, such as dogs, cats, horses.

5. The composition according to claim 1, for use in the treatment and prophylaxis of diseases selected from vulvo-vaginitis, recurrent urethral infections and infections of the lower urinary tract, recurrent infections of the respiratory tract, and allergic rhinitis.

6. The composition according to claim 1, for use in the treatment of cutaneous-mucosal wounds, striae, wrinkles, cracked and excoriated skin, onychomycosis, corneal microtrauma in contact lenses wearers.

7. The composition according to claim 1, for use in the treatment and prophylaxis of tonsillitis and pharyngitis, Candida albicans infections, Herpes Virus Simplex 1 and 2 cutaneous-mucosal lesions, acne lesions and folliculitis.

8. The composition according to claim 1, for use in the treatment of compression ulcers and diabetic ulcers, infections in immune-compromised patients, infections in hospitalized elderly patients.

9. The composition according to claim 1, for use in the treatment of inflammatory micro-environment.

10. A process for the preparation of the pharmaceutical composition as defined in claims 1-9, comprising the mixing under humid conditions of cellular extracts of Saccharomyces cerevisiae and of Lactobacillus acidophilus separately obtained by homogenization of aqueous suspensions of cells of Lactobacillus acidophilus and by homogenization of aqueous suspensions of cells of Saccharomyces cerevisiae followed by extraction with sodium hydroxide.

11. Use of a mixture of cellular extracts of Saccharomyces cerevisiae and of Lactobacillus acidophilus, alone or in combination with other active principles, for the treatment and prophylaxis of diseases of infectious aetiology.
